(19) United States
(12) Patent Application Publication
(10) Pub. No.: US 2020/0197645 A1
(43) Pub. Date: Jun. 25, 2020

(54) CUSHION MEMBER HAVING A PRE-STRESSED ELEMENT AND METHOD OF MANUFACTURE

(71) Applicant: KONINKLIJKE PHILIPS N.V., Eindhoven (NL)

(72) Inventor: ROBERT WILLIAM BAIKO, PITTSBURGH, PA (US)

(21) Appl. No.: 16/712,575

(22) Filed: Dec. 12, 2019

Related U.S. Application Data
(60) Provisional application No. 62/783,234, filed on Dec. 21, 2018.

(51) Int. Cl. A61M 16/06

(52) U.S. Cl. CPC A61M 16/0683; A61M 16/0622

(57) ABSTRACT

A cushion member is for a patient interface device. The cushion member includes a body portion, a patient contacting portion, and a pre-stressed element located in at least one of the body portion or the patient contacting portion.

### CROSS-REFERENCE TO RELATED APPLICATIONS

This patent application claims the priority benefit under 35 U.S.C. § 119(e) of U.S. Provisional Application No. 62/783,234, filed on Dec. 21, 2018, the contents of which are herein incorporated by reference.

### BACKGROUND OF THE INVENTION

### 1. Field of the Invention

The present invention pertains to cushion members, in particular cushion members including pre-stressed elements, for patient interface assemblies, such as patient interface assemblies used in delivering pressurized breathing gas to the airway of a patient. The present invention also pertains to patient interface assemblies including such cushion members. The present invention also pertains to methods of manufacturing cushion members for patient interface assemblies.

### 2. Description of the Related Art

Many individuals suffer from disordered breathing during sleep. Sleep apnea is a common example of such sleep disordered breathing suffered by millions of people throughout the world. One type of sleep apnea is obstructive sleep apnea (OSA), which is a condition in which sleep is repeatedly interrupted by an inability to breathe due to an obstruction of the airway; typically the upper airway or pharyngeal area. Obstruction of the airway is generally believed to be due, at least in part, to a general relaxation of the muscles which stabilize the upper airway segment, thereby allowing the tissues to collapse the airway. Another type of sleep apnea syndrome is a central apnea, which is a cessation of respiration due to the absence of respiratory signals from the brain's respiratory center. An apnea condition, whether obstructive, central, or mixed, which is a combination of obstructive and central, is defined as the complete or near cessation of breathing, for example a 90% or greater reduction in peak respiratory air-flow.

Those afflicted with sleep apnea experience sleep fragmentation and complete or nearly complete cessation of ventilation intermittently during sleep with potentially severe degrees of oxyhemoglobin desaturation. These symptoms may be translated clinically into extreme daytime sleepiness, cardiac arrhythmias, pulmonary-artery hypertension, congestive heart failure and/or cognitive dysfunction. Other consequences of sleep apnea include right ventricular dysfunction, carbon dioxide retention during wakefulness, as well as during sleep, and continuous reduced arterial oxygen tension. Sleep apnea sufferers may be at risk for excessive mortality from these factors as well as by an elevated risk for accidents while driving and/or operating potentially dangerous equipment.

Even if a patient does not suffer from a complete or nearly complete obstruction of the airway, it is also known that adverse effects, such as arousals from sleep, can occur where there is only a partial obstruction of the airway. Partial obstruction of the airway typically results in shallow breathing referred to as a hypopnea. A hypopnea is typically defined as a 50% or greater reduction in the peak respiratory air-flow. Other types of sleep disordered breathing include, without limitation, upper airway resistance syndrome (UARS) and vibration of the airway, such as vibration of the pharyngeal wall, commonly referred to as snoring.

It is well known to treat sleep disordered breathing by applying a continuous positive air pressure (CPAP) to the patient's airway. This positive pressure effectively “splints” the airway, thereby maintaining an open passage to the lungs. It is also known to provide a positive pressure therapy in which the pressure of gas delivered to the patient varies with the patient's breathing cycle, or varies with the patient's breathing effort, to increase the comfort to the patient. This pressure support technique is referred to as bi-level pressure support, in which the inspiratory positive airway pressure (IPAP) delivered to the patient is higher than the expiratory positive airway pressure (EPAP). It is further known to provide a positive pressure therapy in which the pressure is automatically adjusted based on the detected conditions of the patient, such as whether the patient is experiencing an apnea and/or hypopnea. This pressure support technique is referred to as an auto-titration type of pressure support, because the pressure support device seeks to provide a pressure to the patient that is only as high as necessary to treat the disordered breathing.

Pressure support therapies as just described involve the placement of a patient interface device including a mask component having a soft, flexible sealing cushion member on the face of the patient. The mask component may be, without limitation, a nasal mask that covers the patient's nose, a nasal/oral mask that covers the patient's nose and mouth, or a full face mask that covers the patient's face. Such patient interface devices may also employ other patient contacting components, such as forehead supports, cheek pads and chin pads. The patient interface device is typically secured to the patient's head by a headgear component. The patient interface device is connected to a gas delivery tube or conduit and interfaces the pressure support device with the airway of the patient, so that a flow of breathing gas can be delivered from the pressure/flow generating device to the airway of the patient.

Cushion members for patient interface assemblies are typically created in a single molding process, where the shape of the cushion member is retained after being removed from the mold. Patient contacting portions commonly lack internal stresses. As a result, when the patient contacting portions engage the face of the patient, they often buckle easily when exposed to small amounts of compression, thus providing locations where leaks might occur.

### SUMMARY OF THE INVENTION

Accordingly, it is an object of the present invention to provide a cushion member for a patient interface assembly. The cushion member comprises a body portion, a patient contacting portion, and a pre-stressed element disposed in at least one of the body portion or the patient contacting portion.

It is another object of the present invention to provide a patient interface assembly comprising a frame, a circuit connector connected to the frame, a number of headgear connectors coupled to the frame, and the aforementioned cushion member.

It is yet another object of the present invention to provide a method of manufacturing a cushion member for a patient interface assembly. The cushion member has a body portion and a patient contacting portion. The method comprises positioning an element on a first molding tool, the element being positioned on the first molding tool such that at least a portion of the element is in a stressed state; positioning a second molding tool with respect to the first molding tool such that a cavity is defined between the first molding tool and the second molding tool, wherein the at least a portion of the element is disposed within the cavity; providing a quantity of material in the cavity to form an overlying member bonded to the element, the overlying member and the element forming the cushion member; and removing the cushion member from the first and second molding tools, the at least a portion of the element being located in the patient contacting portion.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIGS. 1-4, respectively, are front left, front right, back left, and back right isometric simplified views of a cushion member and a pre-stressed element for the same, in accordance with one non-limiting embodiment of the disclosed concept;

FIG. 5A is a front left isometric view of the element of FIGS. 1-4;

FIG. 5B is an enlarged view of the portion of the element of FIG. 5A, shown generally at 5B in FIG. 5A;

FIG. 6A shows a front isometric simplified view of a portion of another element, in accordance with another non-limiting embodiment of the disclosed concept;

FIGS. 6B-6D show simplified sectional views of other elements, in accordance with other non-limiting embodiments of the disclosed concept;

FIG. 6E is a side simplified view of a portion of another element, in accordance with another non-limiting embodiment of the disclosed concept;

FIG. 7A shows a partially sectional simplified view of the portion of the element of FIG. 6E located on a first molding tool, shown with a second molding tool spaced from the first molding tool, in accordance with an embodiment of the disclosed concept;

FIG. 7B shows the arrangement of FIG. 7A with the second molding tool engaged with the first molding tool;

FIG. 8A shows a partially sectional simplified view of a portion of the element of FIGS. 1-5B located on another molding tool, shown with a second molding tool spaced therefrom, in accordance with another non-limiting embodiment of the disclosed concept;

FIG. 8B shows the arrangement of FIG. 8A with the second molding tool engaged with the first molding tool;

FIG. 8C shows a section view of a portion of the first molding tool with an adhesive material disposed thereon;

FIG. 9 shows a simplified view of the portion of the element of FIGS. 8A and 8B in a non-stressed state;

FIG. 10 shows a simplified view of the portion of the element of FIG. 9 in a stressed state, shown with the overlying member bonded thereto in dashed line drawing, and corresponding to the position of said elements just before being released from molding tools; and

FIG. 11 shows a simplified view of the portion of the element of FIG. 10 in a stressed state, shown with the overlying member bonded thereto in dashed line drawing, and corresponding to the position of said elements after being released from molding tools.

### DETAILED DESCRIPTION OF EXEMPLARY EMBODIMENTS

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As used herein, the statement that two or more parts or components “engage” one another shall mean that the parts exert a force against one another either directly or through one or more intermediate parts or components. As used herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As employed herein, the term “annular-shaped” shall mean having a substantially centrally disposed through hole. For example, a component that is annular-shaped has a body with a substantially centrally disposed through hole.

As employed herein, the term “stressed state” shall refer to a positioning of an element in which the element is forcibly held in a position which differs from a position in which the element would otherwise be disposed in the absence of externally applied forces. In contrast to “stressed state,” the term “relaxed state” shall refer to a positioning of an element in which the element is naturally disposed and substantially free of any externally applied forces. Components in accordance with the disclosed concept may be in different stressed states. For example and without limitation, a first component may be in a first stressed state when a first set of externally applied forces forcibly hold the first component in a first position, whereas a second component may be in a second, different stressed state when a second set of externally applied forces different than the first set of forces forcibly hold the second component in a second position.

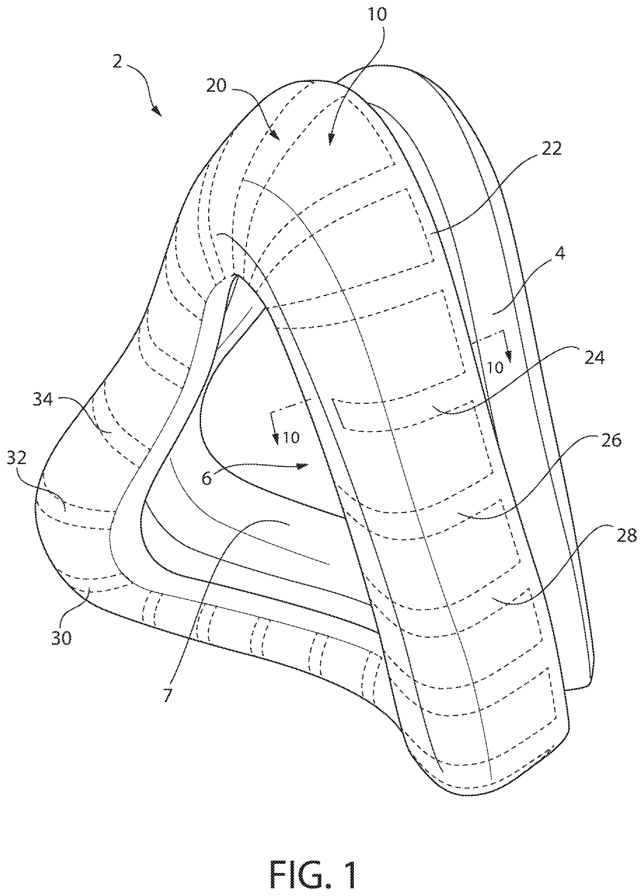

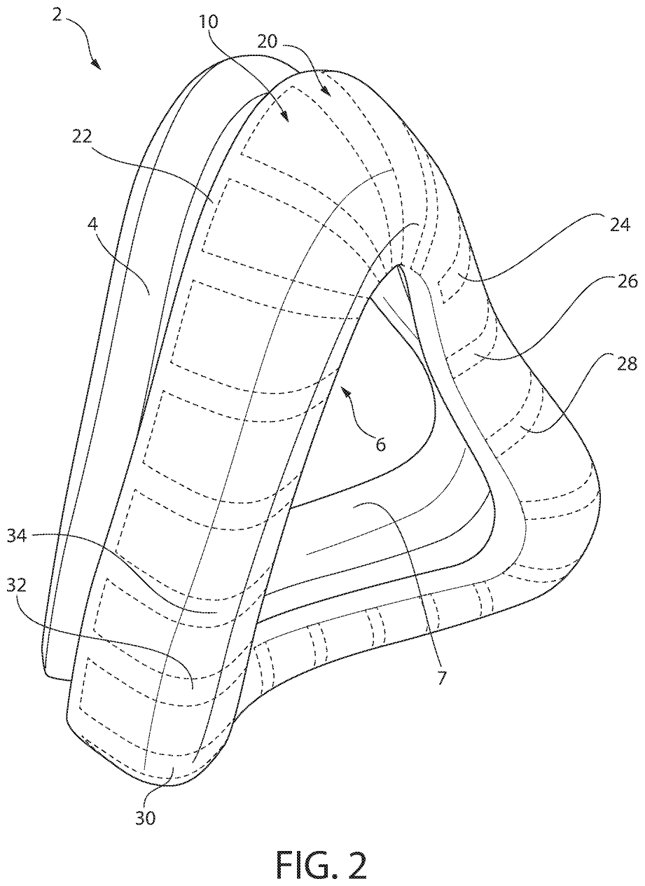

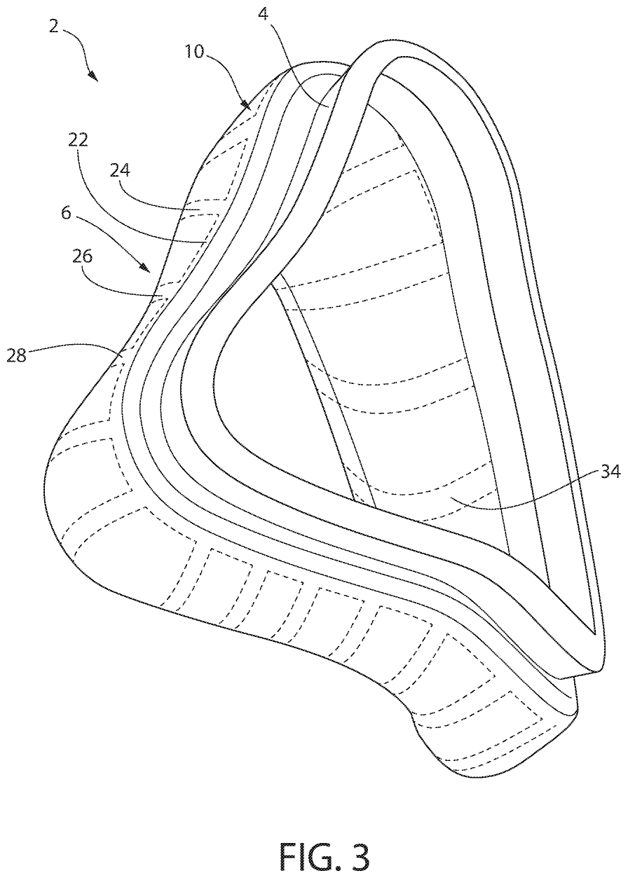

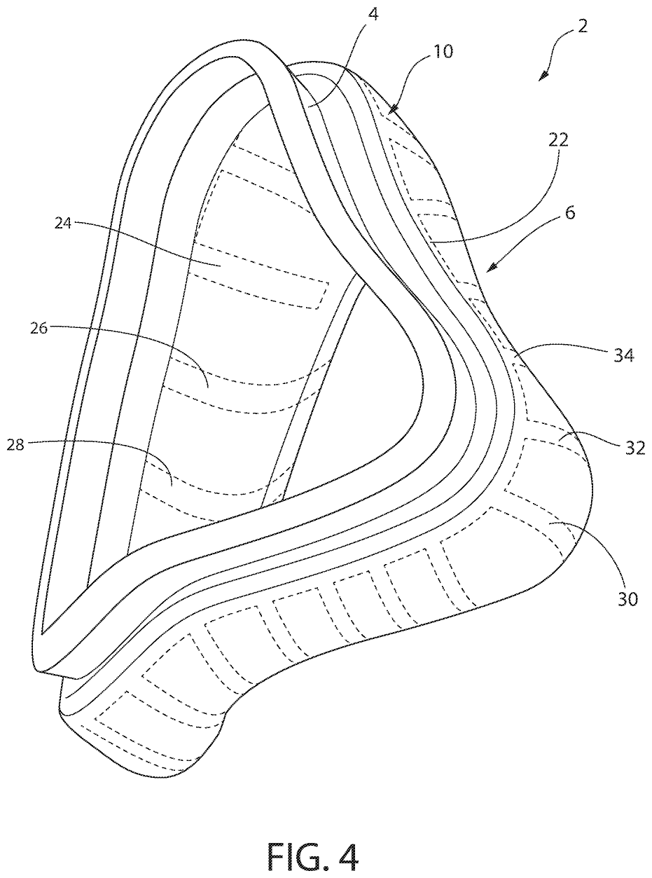

FIGS. 1-4 are different isometric views of a cushion member 2, in accordance with one non-limiting embodiment of the disclosed concept. As shown in FIG. 1, cushion member 2 has a body portion 4 and a patient contacting portion 6 extending from body portion 4. In one embodiment, patient contacting portion 6 includes a sealing flap, and extends generally radially inwardly from body portion 4 toward a centrally disposed thru hole 7. Thru hole 7 passes through body portion 4 and patient contacting portion 6. In operation, patient contacting portion 6 is configured to engage and seal against the face of a patient during pressure support therapy. As such, body portion 4 and patient contacting portion 6 each define thru hole 7 through which breathing gas is communicated from a gas flow generator (not shown) and hose (not shown) to the airway of the patient. Cushion member 2 can generally be stated as including an overlying member 10 and a pre-stressed element 20 (shown in hidden line drawing in FIGS. 1-4). In one example embodiment overlying member 10 is bonded to pre-stressed element 20.

In one example embodiment, overlying member 10 is a first unitary component made from a first single piece of material, and pre-stressed element 20 may be a second unitary component made from a second single piece of material different than the first single piece of material. In another example embodiment, the overlying member and the pre-stressed element may be made of the same piece of material. The first and second pieces of material of overlying member 10 and pre-stressed element 20, respectively, may be any suitable material known in the art (e.g., without limitation, silicone). Furthermore, pre-stressed element 20 may, in an example embodiment, be of a greater rigidity than overlying member 10. It will be also be appreciated that a suitable alternative overlying member and a suitable alternative pre-stressed element, without departing from the scope of the disclosed concept, may each, alone or in combination, be made of multiple materials.

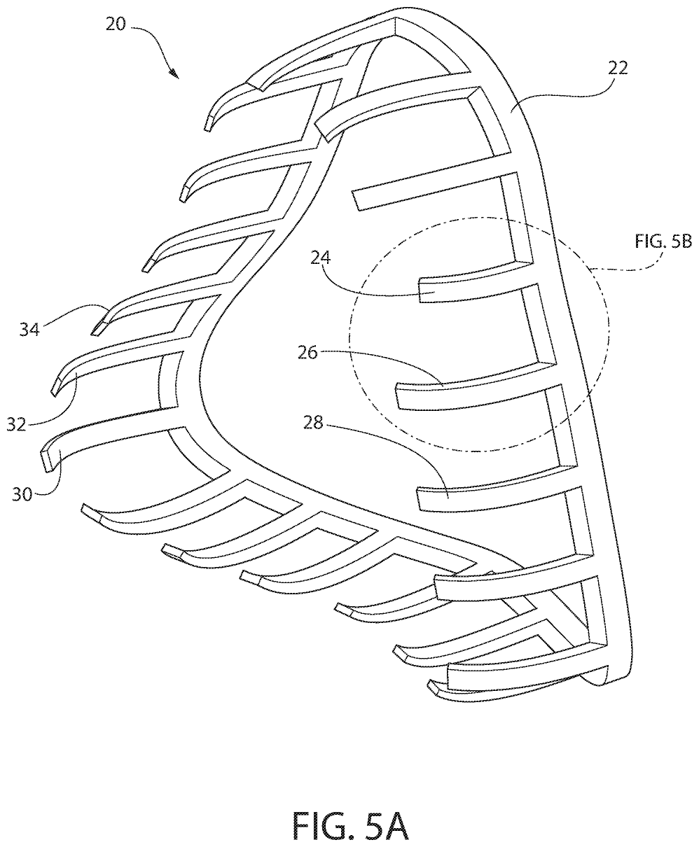

FIG. 5A shows a front isometric view of element 20 without overlying member 10. Element 20 may be made via any suitable method (e.g., without limitation, injection molding). In one example embodiment, pre-stressed element 20 includes a frame member having a support portion 22 and a plurality of projections (e.g., rib members 24,26,28,30,32,34) extending outwardly from support portion 22. In the exemplary embodiment, support portion 22 is annular-shaped and is generally structured to surround the nose and mouth of the patient. It will, however, be appreciated that a suitable alternative support portion need not be annular-shaped, but instead may be structured to only be disposed about a localized region of the face of the patient proximate the airway, without departing from the scope of the disclosed concept. Additionally, in accordance with the disclosed concept, rib members 24,26,28,30,32,34 are structured to be located in patient contacting portion 6 of cushion member 2. See, for example, FIGS. 1-4.

FIG. 5B is an enlarged view of a portion of FIG. 5A. As shown, a first rib member 24 and a second rib member 26 each have respective first and second overall lengths 44,46 from support portion 22 to respective distal ends 25,27 thereof. Additionally, first overall length 44 is different than (e.g., less than) second overall length 46. Referring again to FIG. 5A, it is contemplated that any of rib members 28,30,32,34 may be of any suitable length, be made of any suitable different material, and/or have any suitable different shape/geometry, without departing from the scope of the disclosed concept.

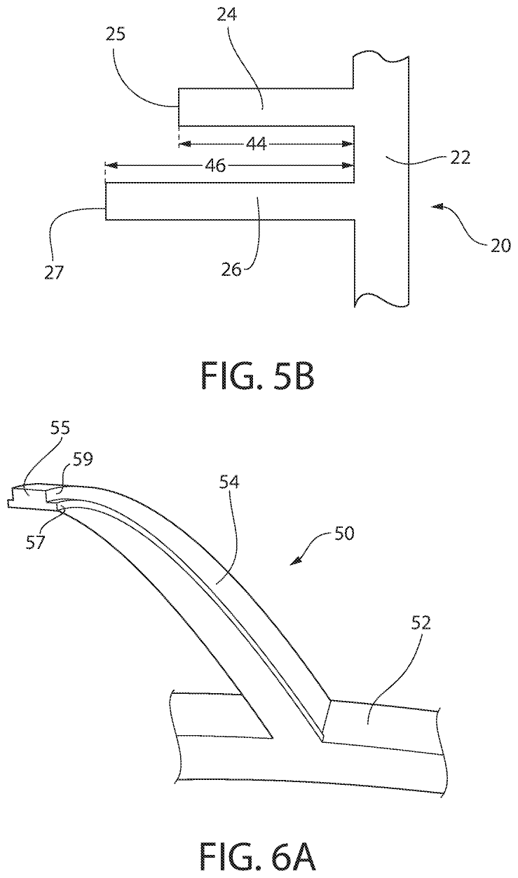

For example and without limitation, FIGS. 6A-6E show portions of different elements 50,60,70,80120 that may be used in cushion member 2 in place of element 20. As shown in FIG. 6A, a rib member 54 extends from a support portion 52 and has a distal end 55. Rather than having a generally rectangular-shaped cross-section, as provided for with rib members 24,26,28,30,32,34 (see FIG. 5A), rib member 54 has a profile comprised of a relatively wide rectangular-shaped base portion 57 and a relatively narrow rectangular-shaped top 59 extending from a central portion thereof. FIG. 6B shows a cross-section 65 of rib member 60 which is trapezoid-shaped. FIG. 6C shows a cross-section 75 of rib member 70 having a relatively wide rectangular-shaped base member 77 and a relatively narrow rectangular-shaped top 79 extending from the top of base member 77. FIG. 6D shows a rectangular-shaped cross-section 85 of rib member 80 substantially similar to rib members 24,26,28,30,32,34 of FIGS. 1-4. FIG. 6E shows a portion of an element 120 having a support portion 122 and a rib member 124 extending therefrom. Rib member 124 includes a distal end portion 125 and a proximal end portion 127 located adjacent support portion 122. In one example embodiment, when element 120 is substituted into cushion 2 for element 20, it will be appreciated that proximal end portion 127 is located at or about body portion 4 of cushion member 2. Additionally, as shown, distal end portion 125 of rib member 124 has an aperture 129, the purpose of which will be discussed below.

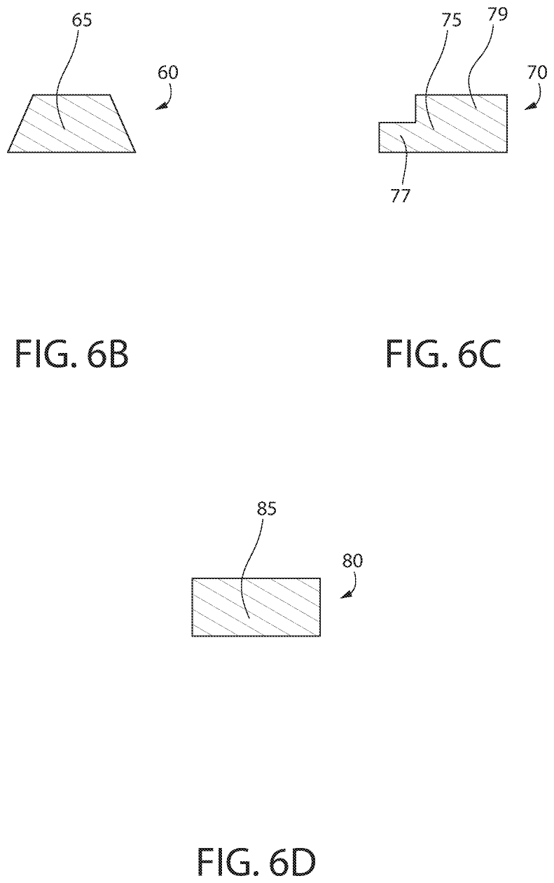

FIGS. 7A and 7B schematically illustrate a method by which a cushion member (e.g., cushion member 2 including element 120 in place of element 20) may be manufactured. As shown in FIGS. 7A and 7B, rib member 124 of element 120 is in a different position than as provided for in FIG. 6E. Specifically, in FIG. 6E rib member 124 is in a non-stressed state. However, in accordance with the disclosed concept, during manufacturing rib member 124 is positioned in a stressed state, i.e., is biased away from the non-stressed state. Compare, for example, the position of rib member 124 in FIG. 6E versus FIGS. 7A and 7B. That is, as shown in FIGS. 7A and 7B, rib member 124 is forcibly bent around a portion (not numbered) of a first molding tool 202.

Continuing to refer to FIG. 7A, the method may include the steps of positioning element 120 on first molding tool 202 such that each of the rib members (only one rib member 124 is shown in FIG. 7A, but see, for example, rib members 24,26,28,30,32,34 of element 20 in FIG. 5A) is in a stressed state. In one example embodiment, first molding tool 202 is provided with a pin member 203 which is structured to extend into aperture 129 (FIG. 6E) of rib member 124 in order to retain rib member 124 on first molding tool 202 in a stressed state. It is to be appreciated, however, that other suitable retention mechanism or mechanisms may be employed, without varying from the scope of the present invention. For example and without limitation, each of the additional elements 50,60,70,80 in FIGS. 6A-6D could be retained on a different first molding tool by virtue of the geometries of their rib members, as shown and described herein. A rib member (e.g., one of rib members 24,26,28,30,32,34, or rib members of elements 50,60,70,80) may be maintained on a first molding tool by any suitable mechanism including adhesive connections, tongue and groove connections, vacuum forces, retaining clips, button snaps, and/or magnets.

The method further includes positioning a second molding tool 212 with respect to first molding tool 202 such that a cavity (e.g., cavity 232 shown in FIG. 7B, and formed by first molding tool 202 engaging second molding tool 212) is defined between first molding tool 202 and second molding tool 212. As shown in FIG. 7B, rib member 124 is located in cavity 232. It will be appreciated that any suitable number of the rib members (e.g., rib members 24,26,28,30,32,34 of element 20 in FIG. 5A) may be provided in a cavity between first and second molding tools. After the first and second molding tools 202 and 212 are positioned as shown in FIG. 7B, a quantity of material is provided in cavity 232 to form overlying member 10 bonded to element 120. In one example embodiment, the material is provided by injecting the quantity of material into cavity 232 (i.e., via an injection molding process). Accordingly, overlying member 10 of cushion member 2 may be overmolded onto elements 20,50,60,70,70,120. The method may also include maintaining element 120 on first molding tool 202 during the injecting step. The method further includes removing cushion member (e.g., cushion member 2 including element 120 instead of element 20) from first and second molding tools 202,212.

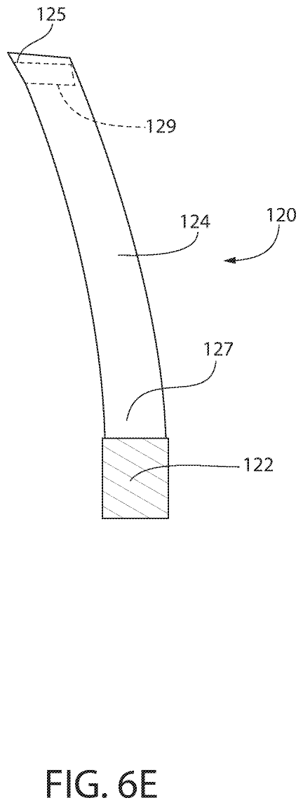

FIG. 8A shows a portion of element 20 positioned on a first molding tool 302, in accordance with another example embodiment of the present invention. As shown, rib member 24 is in a non-stressed state. Accordingly, the method of manufacturing cushion member 2 may further include moving rib member 24 toward first molding tool 302 by either moving a second molding tool 312 toward first molding tool 302, or moving first molding tool 302 toward second molding tool 312, or moving both first and second molding tools 302,312 toward each other. See, for example, FIG. 8B, which shows first and second molding tools 302,312 engaged with each other, and a cavity 332 formed therebetween.

Furthermore, FIG. 8C shows a section view of first molding tool 302 with a quantity of adhesive material 303 disposed thereon. It will be appreciated that a rib member, e.g., rib member 24, may be bent, manually or otherwise, and disposed on adhesive material 303. After molding, rib member 24 may be removed from first molding tool 302, together with the rest of cushion member 2.

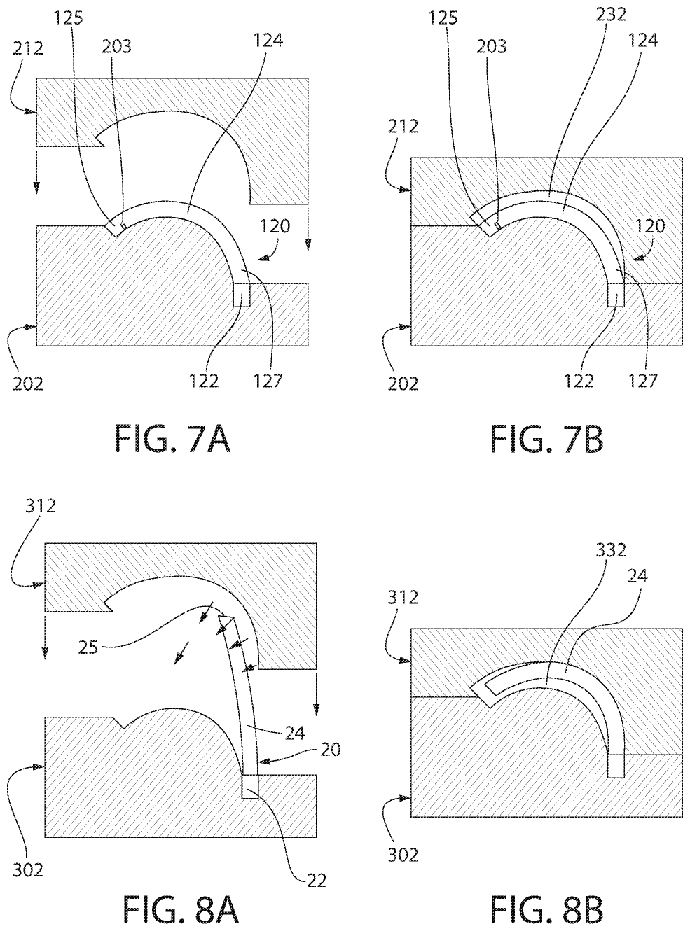

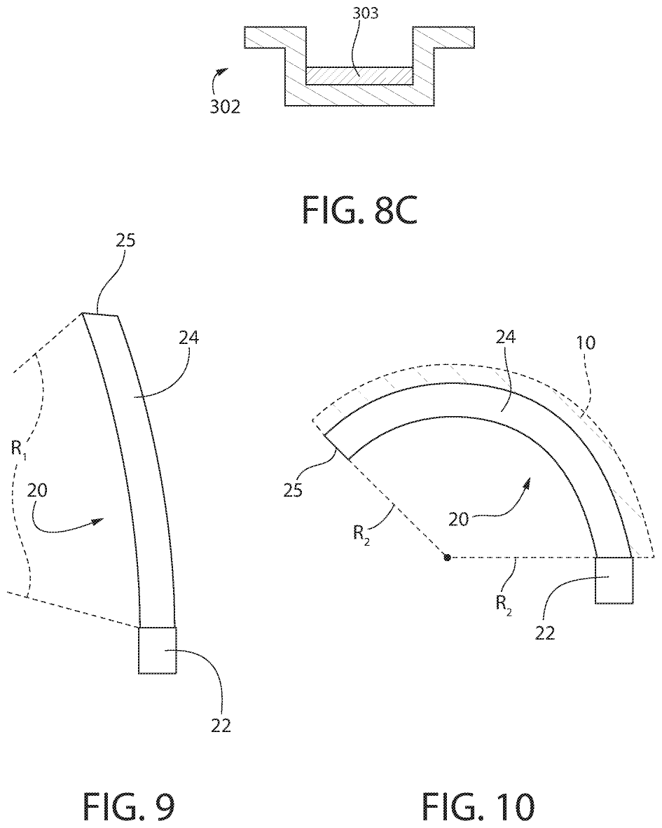

From the foregoing, it is thus to be appreciated that manufacturing cushion member 2 in the manner described herein provides for rib members 24,26,28,30,32,34, and rib members of elements 50,60,70,80,120, being in stressed states. That is, when cushion member 2 is not donned by a patient, rib members 24,26,28,30,32,34, and rib members of elements 50,60,70,80,120 may each be in stressed states. For example, FIG. 9 shows rib member 24 in a non-stressed state before being manufactured with overlying member 10 to form cushion member 2, whereas FIG. 10 shows rib member 24 in a stressed state after having been manufactured with overlying member 10 (shown in dashed line drawing in FIG. 10) to form cushion 2. By comparing the radii of curvature in FIGS. 9 and 10, R₁ and R₂, respectively, it can be appreciated that in FIG. 10, distal end 25 is biased away from support portion 22 and toward the relaxed, and non-stressed, position of FIG. 9. Stated differently, when in the stressed state, distal end 25 of element 20 is biased away from body portion 4.

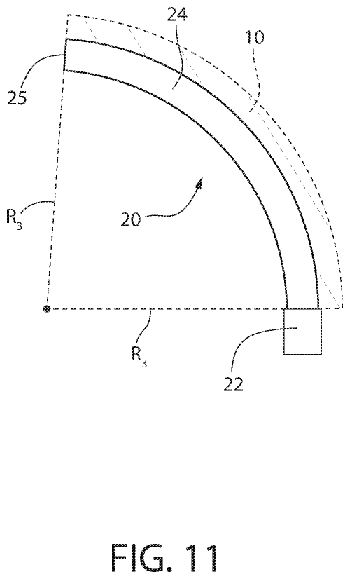

Moreover, in one example embodiment rib member 24 has a radius of curvature R₂ when rib member 24 is located within cavity 332 (FIG. 8B) and a radius of curvature R₃, see FIG. 11, when the cushion member is removed from first and second molding tools 302,312, radius of curvature R₃ being different than radius of curvature R₂. Thus, it will be appreciated that rib member 24, when released from molding tools 302,312, begins to move toward its non-stressed state (e.g., the position shown in FIG. 9), but is prevented from reaching said state by overlying member 10. Additionally, while the disclosed concept has been described in association with a rib member being generally planar in a non-stressed state and curved (e.g., non-planar) in a stressed state, it is within the scope of the disclosed concept to have a suitable alternative element be generally curved (e.g., non-planar) in a non-stressed state and planar in a stressed state.

By having rib members in stressed states located in patient contacting portion 6, cushion member 2 is advantageously provided with a mechanism to resist buckling and/or to resist the collapse of the patient contacting portion during therapy. This scenario, in prior art cushion members (not shown), results in a relatively weak seal wherein leaks can occur, thus compromising pressure support therapy. However, in accordance with the disclosed concept, when cushion member 2 is donned by the patient, rib members 24,26,28,30,32,34, and rib members of elements 50,60,70,80,120 exert a relatively strong force on the face of the patient, a force which tends to maintain the structure of patient contacting portion 6.

For example, and with reference again to FIG. 1, when cushion member 2 is not donned by a patient (e.g., as currently shown), rib members 24,26,28,30,32,34 are already in a stressed state and thus are biased such that rib members 24,26,28,30,32,34 generally want to straighten in order to return to the positioning shown in FIG. 5A. Furthermore, it can be appreciated that patient contacting portion 6 may be in a first stressed state and pre-stressed element 20 may be in a second stressed state different than the first stressed state. When cushion member 2 is actually donned by the patient, rib members 24,26,28,30,32,34, and rib members of elements 50,60,70,80,120, move to an even more stressed state. Specifically, distal ends of rib members 24,26,28,30,32,34, and rib members of elements 50,60,70,80,120, move more toward the interior of cushion member 2, toward body portion 4, and away from the non-stressed state. The cushion member being comprised of a stressed component and non-stressed component will provide support not possible with a single cushion member molded in a non-stressed state.

While the disclosed concept has been described herein in association with a full-face cushion member 2, it will be appreciated that suitable alternative cushion members may be manufactured by the same method as provided herein, and thus possess the same advantages. Such cushion members include nasal cushion members and pillows style cushion members. Additionally, while rib members 24,26,28,30,32,34 of element 20 generally extend around annular-shaped support portion 22, it will be appreciated that a suitable alternative element may be provided with any number of rib members, and at any location(s) along support portion 22. For example, if a user desires increased support at only specific locations along a patient contacting portion, a cushion member may include an element having rib members located only proximate the specific location along the patient contacting portion. Furthermore, it is contemplated that different rib members may have different material properties. For example, rib member 24 may have a first stiffness, and rib member 26 may have a second stiffness different than the first stiffness. If the second stiffness of rib member 26 is greater than the first stiffness of rib member 24, than the patient contacting portion, while still being supported by the rib members of element 20, will be provided with more support proximate rib member 26 than rib member 24.

Additionally, it will be appreciated that a patient interface assembly may be provided, in accordance with the disclosed concept. The patient interface assembly may include a frame, a circuit connector connected to the frame, a number of headgear connectors coupled to the frame, and cushion member 2.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A cushion member for a patient interface assembly, the cushion member comprising:
a body portion;
a patient contacting portion; and
a pre-stressed element disposed in at least one of the body portion or the patient contacting portion.

2. The cushion member of claim 1, wherein the pre-stressed element has a first portion disposed in or on the body portion and a second portion disposed in or on both of the body portion and the patient contacting portion.

3. The cushion member of claim 2, wherein the patient contacting portion comprises a sealing flap.

4. The cushion member of claim 2, wherein the patient contacting portion is in a first stressed state; and wherein the pre-stressed element is in a second stressed state different than the first stressed state.

5. The cushion member according to claim 1, wherein the pre-stressed element comprises a frame member having a support portion and a plurality of projections extending outwardly from the support portion and disposed in the patient contacting portion.

6. The cushion member according to claim 5, wherein at least one of the plurality of projections is in a stressed state when the cushion member is not donned by the patient.

7. The cushion member according to claim 6 wherein each of the plurality of projections is in a stressed state when the cushion member is not donned by the patient.

8. The cushion member according to claim 5, wherein the support portion is annular-shaped.

9. The cushion member according to claim 1, wherein the body portion and the patient contacting portion are an overlying member overmolded onto the pre-stressed element.

10. The cushion member according to claim 9, wherein the overlying member is a first unitary component made from a first single piece of material; and wherein the pre-stressed element is a second unitary component made from a second single piece of material different than the first single piece of material.

11. The cushion member according to claim 9, wherein the pre-stressed element is of a greater rigidity than the overlying member.

12. A patient interface assembly comprising:
a frame;
a circuit connector connected to the frame;
a number of headgear connectors coupled to the frame; and
a cushion member comprising:
a body portion coupled to the frame,
a patient contacting portion, and
a pre-stressed element disposed in at least one of the body portion or the patient contacting portion.

13. A method of manufacturing a cushion member for a patient interface assembly, the cushion member having a body portion and a patient contacting portion, the method comprising:
positioning an element on a first molding tool, the element being positioned on the first molding tool such that at least a portion of the element is in a stressed state;
positioning a second molding tool with respect to the first molding tool such that a cavity is defined between the first molding tool and the second molding tool, wherein the at least a portion of the element is disposed within the cavity;
providing a quantity of material in the cavity to form an overlying member bonded to the element, the overlying member and the element forming the cushion member; and
removing the cushion member from the first and second molding tools, the at least a portion of the element being disposed in the patient contacting portion.

14. The method according to claim 13, wherein providing a quantity of material in the cavity comprises injecting the quantity of material into the cavity.

15. The method according to claim 14, further comprising maintaining the element on the first molding tool during injecting.

16. The method according to claim 13, wherein the element comprises a frame member having a support portion and a plurality of projections extending outwardly from the support portion; wherein the at least a portion of the element comprises at least one of the plurality of projections; and wherein the at least one of the plurality of projections has a first radius of curvature (R₂) when the least one of the plurality of projections is disposed within the cavity and a second radius of curvature (R₃) when the cushion member is removed from the first and second molding tools, the second radius of curvature being different than the first radius of curvature.

17. The method according to claim 13, further comprising moving the at least one of the plurality of projections toward the first molding tool by moving one or both of the first molding tool and the second molding tool toward the other of the first molding tool and the second molding tool.
